(19) United States
(12) Patent Application Publication
(10) Pub. No.: US 2009/0013993 A1
(43) Pub. Date: Jan. 15, 2009

(54) Inhalation Device

(75) Inventors: Jonathon James Bird, Port Melbourne (AU); Robert James Bird, Port Melbourne (AU); Vicki Kathleen Bird, Port Melbourne (AU); Nicholas James Bird, Port Melbourne (AU)

Correspondence Address: HONIGMAN MILLER SCHWARTZ & COHN LLP, 38500 WOODWARD AVENUE, SUITE 100, BLOOMFIELD HILLS, MI, 48304-5048, US

(73) Assignee: Bird Healthcare Pty. Ltd., Port Melbourne (AU)

(21) Appl. No.: 12/163,525

(22) Filed: Jun. 27, 2008

(30) Foreign Application Priority Data
Jun. 29, 2007 (AU) 2007903524

(51) Int. Cl. A61M 11/00

(52) U.S. Cl. 128/200.15; 128/200.23

(57) ABSTRACT

A dosage device capable of delivery of a full dosage of medicament to a person in need of treatment through the mouth comprising a storage chamber for more or less temporarily storing the medicament after receiving the medicament from a suitable dispenser, such as an MDI inhaler, and a mouthpiece having a sealing ring for sealing the mouthpiece within the mouth of the person using the dosage device connected to the storage chamber in which a one way valve is located intermediate the storage chamber and mouthpiece to prevent air exhaled by the person from re-entering the storage chamber to reduce or prevent the risk of contamination or cross-contamination occurring within the storage chamber thereby allowing repeated use of the dosage device even by different persons, without the absolute need to clean the dosage device after each use.

### CROSS-REFERENCE TO RELATED APPLICATIONS

This application claims priority to Australian Patent Application No. 2007903524, filed Jun. 29, 2007, the entirety of which is incorporated by reference herein.

### FIELD

The present invention relates generally to inhalation devices for use in delivering a measured dose of medicament to the airways of a person suffering from a bronchial condition and to methods of using such devices to administer the medicament.

### BACKGROUND

In many situations it is necessary to deliver a full dose of medication or medicament to a person suffering from a breathing condition or lung complaint through the air passages or airways of a person, such as for example, during the treatment of a bronchial condition, such as for example asthma and other respiratory conditions, or when conducting lung function testing or monitoring in diagnosing a condition or ailment or when a person is undergoing treatment for a respiratory condition. One example of devices for delivering dosages of medicaments through the airways of a person is an inhaler. Inhalers are used to provide medication for bronchial conditions, such as for example, inhalers are the main treatment for asthma. Inhalers come in a variety of different styles and types for slightly different purposes and for administering different medicaments. However, using an inhaler can be difficult for some people as it requires coordination to produce a “puff” or metered dose to deliver the medicament to the person. One type of inhaler is a Metered Dose Inhaler (MDI) which is used to deliver various types and brands of medicaments directly to and through the mouth or oral cavity to the lungs of a person suffering from this condition. The MDI contains a pressurised inactive gas that propels a dose of medication directly to the airway of a person in each “puff” or discharge of the MDI. Each dose is dispensed by being released under pressure from the MDI by pressing the top of the inhaler. Although this type of inhaler is quick to use, small and convenient to carry, it is not all that easy to use since it requires good coordination to be effective as it is necessary to both press the canister and inhale fully simultaneously to deliver the full measured dose into the lungs. Thus, there is a need for an inhalation device that is easier to use, requiring less coordination, between activating the inhaler and actually inhaling.

For a variety of reasons, such as lack of coordination, often when using an inhaler the full dose does not enter the airways or is not delivered to the lungs but rather escapes, “leaks” or flows away from the mouth without reaching the air passages, such as through the edges of the mouth not being tightly sealed to the MDI dispenser or by the lips not forming a sufficient seal around the MDI to contain all of the dose within the oral cavity or through premature opening of the mouth. Thus, a reduced amount of medicament is often administered to the lungs, which is less than ideal particularly when a person suffers from acute asthma and requires immediate administration of the full dose for relief from this debilitating condition. Thus, there is a need for more reliable administration of a medicament using an inhalation device.

One attempt to overcome the problem of a reduced dose of medicament entering the air ways due to a lack of coordination using a MDI is to provide a reservoir or spacer for temporarily storing the full dose discharged from the MDI into the reservoir so that the full dose can be withdrawn from the reservoir into the mouth by inhaling as a separate action to dispensing the medication from its container after pressing the top of the MDI to release the dose to the reservoir thereby reducing the dependency on excellent coordination to administer the medicament. However, one problem of using a reservoir or spacer within which to accumulate or hold a dose prior to actual administration to the lungs is that it is possible after introducing the medication into the air passages of the person during exhaling, to breathe back into the reservoir thereby contaminating the reservoir by the exhaled breath, particularly if the person using the device is suffering from some medical condition, such as for example, an infection or the like. To avoid cross-contamination between subsequent uses of the device, it was necessary to clean the reservoir or spacer after every use in an attempt to lessen the chance of contamination of the device or cross-contamination between the different people using the device. However, effective cleaning of the reservoir or spacer was not always possible, and even if effective cleansing was possible, such cleaning was always time consuming and expensive even when ineffective because of the complexity of the device. Thus, it is desirable to provide an inhalation device for delivering the full dose of medicament which is subject to a reduced risk of contamination. Further it is desirable to provide an inhalation device that does not require cleaning after a single use to prevent contamination or cross-contamination.

Therefore, there is a need to provide a delivery system and/or dosage device which has a reduced chance of being contaminated, or cross-contaminated by exhaling into the device after administering a dose of medication.

Also, existing inhalation systems are expensive if they can only be used once before being discarded, and complex if they need to be cleaned, which makes them expensive to produce and to use which cost is passed onto consumers and/or deprives people of treatment in some circumstances. Thus, there is a need for an effective inhalation device which is less costly to make and use. Further, there is a need for an inhalation device which has a reduced risk of being contaminated so that it can be used repeatedly.

Also, in order to be able to deliver the full dose of medicament from the reservoir there is a need for a modified mouthpiece attached to or attachable to the reservoir, which mouthpiece is adapted to more closely conform to the shape of the mouth so as to provide an improved seal between the mouth and/or lips and the mouthpiece so that there is little or no leakage from the mouth through the corners of the mouth or past the lips during inhaling of the medicament.

Accordingly, it is one aim of the present invention to provide a delivery system and/or dosage device in which expelled air is not withdrawn into the dosage compartment of the device but rather expelled air is directed away from or is discharged from the device, such as by being vented to atmosphere, without entering the reservoir that is being used for temporarily storing the medicament on exhaling into the device after administration of the medicament to the airways of the person through the mouth.

It is another aim of the present invention to provide a mouthpiece which more closely conforms to the shape of the mouth and/or lips of the person using the mouthpiece, so as to reduce the amount of leakage of air between the mouth and the mouthpiece when a person inhales or exhales.

It is another aim of the invention to provide a dosage device or delivery system that is less complex and less expensive to manufacture, assemble and/or delivers a full dose of medicament in a clean and uncontaminated manner that can be used repeatedly.

It has been discovered that a dosage device and/or delivery system can be made where the risk of cross-contamination is considerably reduced by providing a container having vents which allow exhaled breath to escape to atmosphere without passing through the container.

It has been discovered also that a mouthpiece having a sealing profile for engaging with the lips and/or teeth allows an improved seal with the mouth and/or lips to be formed so as to reduce the amount of leakage of airflow past the lips and/or mouth thereby promoting a greater chance that a full dosage of medication can be delivered.

It is to be noted that not all embodiments of the present invention satisfy all aims of the invention. Some embodiments will satisfy one aim, whilst other embodiments will satisfy another aim, and some embodiments may satisfy two or more aims.

### SUMMARY

According to one aspect, there is provided a mouthpiece capable of being used to direct the flow of air including both during inhalation or exhalation comprising a first end provided with a bite element for engaging with the teeth of a person using the mouthpiece to assist in securely maintaining one end of the mouthpiece within the mouth during use of the mouthpiece, and a lip sealing element located at or towards the first end of the mouthpiece for cooperatively engaging with the lips of a person using the mouthpiece to seal the mouthpiece to the mouth so that during use of the mouthpiece there is reduced amount of leakage of air flow from the lips and/or mouth of a person using the mouthpiece to inhale or exhale.

According to another aspect, there is provided a mouthpiece capable of being used with an inhalation device to introduce a medicament to the mouth of a person using the inhalation device through the mouthpiece, the mouthpiece having a first end provided with a bite element for engaging with the teeth of a person using the mouthpiece to assist in securely maintaining the first end of the mouthpiece within the mouth during use and a lip sealing element for cooperatively engaging with the lips of a person to substantially seal the first end of the mouthpiece to the mouth and a second end connected to or connectable to the inhalation device wherein the first end is in fluid communication with a second end so that when the teeth are used to hold the bite element and the lips engage with the lip sealing element to seal the first end of the mouthpiece to the mouth the medicament within the inhalation device can be withdrawn through the mouthpiece into the mouth of the person using the mouthpiece so that substantially all of a dosage of medicament is delivered to the person.

According to another aspect, there is provided a dosage device capable of assisting in delivering a measured dose of a medicament to a person using the dosage device, said dosage device comprising a storage chamber for temporarily storing a measured dose of the medicament after introduction of the medicament into the chamber, the storage chamber provided with an inlet for admitting the measured dose of medicament to the storage chamber and a mouthpiece portion adapted for cooperation with the mouth of a person to withdraw the measured dosage of medicament into and through the mouth of a person upon inhalation by the person wherein the mouthpiece is provided with a bite element for engaging with the teeth of the person using the dosage device to assist in delivering substantially all of the dosage to the person.

According to another aspect, there is provided a mouthpiece for use in connection with an inhalation device comprising at least a delivery tube capable of connection to a source of medicament forming the main body of the mouthpiece, a bite element located at or towards one end of the delivery tube adapted for engagement with the teeth of a person using the mouthpiece and a lip sealing element or profile located around the tube for sealingly engaging with the lips and/or mouth of the person to substantially seal the mouthpiece to the lips and/or mouth wherein the bite element assists in locating the end of the mouthpiece securely within the mouth and the lip sealing element substantially seals the mouth to assist in allowing substantially all of the measured dose of the medicament to be delivered to the airways of a person through the mouth.

According to another aspect, there is provided a delivery system for introducing a medicament into the oral cavity of a person requiring treatment for a bronchial and/or lung condition comprising a storage space for receiving and holding a dose of the medicament prior to introducing the medicament into the airways of the person through the person inhaling wherein the storage space is provided with at least one vent for directing and/or exhausting air exhaled from the person using the device away from the storage space for discharge from the device away from the storage space so as to vent the exhaled breath to atmosphere thereby reducing or eliminating the chance of contaminating the storage space or the contents of the storage space by admitting the exhaled breath to the storage space.

### BRIEF DESCRIPTION OF THE DRAWINGS

The present invention will now be described by way of example with reference to the accompanying drawings in which:

FIG. 1 is a side elevation view of one form of the dosage device of the present invention showing the mouthpiece being connected to the canister.

FIG. 2 is a cross-sectional view of one form of the canister of the dosage device of the present invention showing the profile or configuration of the inner wall of the inlet aperture.

FIG. 2a is an enlarged view of the interconnection between the side wall of the mouthpiece of the device and the side wall of the canister of the device.

FIG. 2b is an enlarged view of the wall of the inlet aperture located in the base of the canister.

FIG. 3 is a plan view of the open end of the canister of FIGS. 1 and 2.

FIG. 4 is an underneath side perspective view of the canister of FIG. 2 showing the single centrally located inlet aperture located in the base of the canister at the inlet end of the canister and the vent slots located circumferentially around in a peripheral flange around the top of the canister.

FIG. 5 is an exploded view of the dosage device of the present invention showing the canister, valve components and mouthpiece of the dosage device in aligned but separated relationship to one another.

FIG. 6 is a cross-sectional view of one form of the mouth piece of the present invention showing an enlarged view of the side of the mouthpiece.

FIG. 6a is an enlarged view of one form of the side wall of the mouthpiece.

FIG. 7 is an underneath or internal view of the mouth piece looking through the mouthpiece.

FIG. 8 is a partially exploded view of the dosage device of the present invention showing the valve arrangement connected to or located within the top of the canister.

FIG. 9 is a partial cross-section of the dosage device illustrating the valve arrangement in the open position showing schematically the flow of air through the dosage device when inhaling through the device.

FIG. 10 is a partial cross-section of the dosage device showing the valve arrangement in a closed position for effecting air outwardly from the dosage device when exhaling through the device.

### DETAILED DESCRIPTION

Referring now to the drawings, illustrative embodiments are shown in detail. Although the drawings represent the embodiments, the drawings are not necessarily to scale and certain features may be exaggerated to better illustrate and explain novel aspects of an embodiment. Further, the embodiments described herein are not intended to be exhaustive or otherwise limit or restrict the claims to the precise form and configuration shown in the drawings and disclosed in the following detailed description.

It is to be noted that use of the expression referring to administration of the medicament or therapeutic agent or pharmaceutical agent to the mouth or oral cavity includes within its scope administration of the medicament etc. to the mouth, oral cavity, throat, lungs, nasal passage, airways, air passages and the like. The use of the word mouth is to make the specification more readable and is not to be taken as limiting of the scope of the invention.

Typically the delivery system or dosage device of the present invention can be of a one piece construction or can be made of two or more individual pieces or subassemblies which can be connected together to form the overall dosage device.

In one form the dosage device or delivery system includes a number of subassemblies. Typically, one of the subassemblies is, or acts as a storage space, chamber, cavity, enclosure, container or similar. Another of the subassemblies includes a valve, a valve arrangement, or components of a valve. A still further subassembly includes a mouthpiece or similar device for administering the medicament to the person. Preferably, there are at least two components which are connectable together to form the dosage device which are a combined storage space and valve arrangement in the one subassembly and a separate mouthpiece as the other assembly. Other combinations are possible.

Typically, the mouthpiece is snap fittingly connected to the storage space to allow assembly of the two subassemblies together to form the dosage device of the present invention. More typically, the valve or valve arrangement is located intermediate the mouthpiece and the storage space.

In one form, the storage space and valve arrangement is formed integrally as a single construction whereas in another form the valve arrangement is removable from the storage space, such as for example, the valve arrangement is a replaceable valve arrangement or a removable valve arrangement fitted to the storage space.

Typically, the valve arrangement is a reusable valve arrangement or is a single use valve arrangement or is a disposable valve arrangement or a multi-use valve arrangement depending upon the particular therapeutic agent, pharmaceutical material, drug or the like being used as the medicament and administered by the device.

Typically, the valve arrangement is a one way valve arrangement allowing movement of air through the valve arrangement in a single direction only. More typically, the valve arrangement allows passage of inhaled air from the storage space but prevents passage of exhaled air through the valve into the storage space after the medicament in the storage space has been administered.

In one form the one way valve includes a web portion for supporting the valve. More typically, the web portion includes a plurality of apertures or similar for allowing passage of air or airflow therethrough when the valve is in an open position. Typically, the apertures are sealed when the valve is in the closed position.

Typically, the one way valve arrangement includes a diaphragm. In one form the diaphragm is one or more flaps. Typically the flap or flaps are movable between an open position in which the flap is deflected or deformed away from the web portion allowing air to move through the valve and a closed position in which the flap is in sealing contact with the web portion so as to prevent flow of air back to the reservoir or storage space.

Typically, the valve in the normal at rest position is in the closed position such that the diaphragm is in contact with the supporting web having the apertures.

Typically, the diaphragm flap of the one way valve is secured or adhered to the centre of the support web of the valve, such as for example, to the hub of the valve at the centre of the apertures. Typically, the flaps are secured to the web by a suitable chemical bonding agent, such as an adhesive, glue, by a thermal bonding, by sonic welding or by a mechanical fastener, such as a rivet, including a plastic rivet or the like.

Typically, the diaphragm flap is a generally circular flat disc, preferably made of a plastics material, typically a polypropylene, such as for example, Tyvek™ made by DuPont. Even more typically, the disc is provided with slits, preferably radially extending slits located at spaced apart locations around the circumference of the disc. More typically, the locations of the slits correspond to the location of the spokes. Even more typically, the slits extend from the edge of the flap radially inwards for a part of the length of the spokes so that the centre portion of the disc is solid and not provided with a slit. Typically, the centre portion is adhered to the web. More typically, the slits divide the circular disc of the flap into segments, sectors, parts, or the like.

Typically, on inhaling the one way valve opens such that the diaphragm flaps are displaced slightly from the supporting web to allow air to flow from the storage space to the mouthpiece and hence into the mouth of the person. More typically on exhaling the flap diaphragm is forced onto the supporting web to close the valve thereby sealing the storage space and preventing exhaled air, fluid, gas or similar from entering the storage space and directing the air flow away from the valve arrangement such as to be discharged or exhausted from the device thereby preventing contamination of the interior of the storage space including the contents of the storage space.

Typically, the one way valve arrangement seats or is locatable within a valve seat provided on or in the storage space, such as for example, the seat, such as for example, a peripheral flange is formed around the perimeter of the storage space. Alternatively, the valve arrangement, particularly the supporting web is fixedly connected to the storage space, such as by chemical bonding using an adhesive, glue, or similar, by thermal bonding using heat to melt or weld the pieces together or sonic welding or similar.

Typically, the storage space is a spacer, reservoir, chamber, cavity, canister, container, vessel, hollow cylinder, or the like enclosure for more or less temporarily storing a dosage of medicament prior to delivery and/or administration. Preferably, the storage space is a hollow spacer in the form of a canister or chamber having tapering sides.

Typically, the interior walls of the dosage device, particularly of the reservoir, mouthpiece or the like, are treated to reduce the attraction of solid particles of medication to the interior walls. In one form, the treatment is a coating, such as a frictionless coating, anti-stick coating, reduced friction coating or the like to assist in the dosage device delivering a full dosage.

In one embodiment the canister is provided with one or more air vents for use in directing air away from the interior of the canister or exhausting air to atmosphere or venting exhaled gases to atmosphere. Typically, there are multiple vents located at spaced apart locations around the edge of the canister. More typically the vents are generally arcuate in shape and/or profile. Preferably, there are five vents located at regularly spaced apart locations around the perimeter of the canister. Even more preferably, the vents are located radially outwards of the valve or of the valve seat, particularly outboard of the flap, to allow expelled air to be directed from the valve to the vents for discharge from the device around the circumference, periphery or outside boundary of the device.

More typically, when the one way valve arrangement is in the closed position, such as the flap diaphragm being forced into contact with the web portion by the pressure of the exhaled breath on the diaphragm to seal the valve, the exhaled breath is directed through the vents for discharge to outside of the canister. Typically, the canister is provided with channels, grooves, ducts, conduits, or the like pathways or guides for providing fluid communication with the interior of the mouthpiece for receiving exhaled air and directing air to the vents for discharge to the outside of the canister.

Typically, the canister is provided with an inlet, typically in the form of an aperture. Typically, the aperture is shaped and/or configured so as to receive the discharge chute or dispensing end of an inhaler. Typically, the inhaler is a Metered Dose Inhaler (MDI), have a shrouded outlet or dispensing port, such as for example, of the conventional type for location within the mouth of a person through which the medicament can be dispensed.

In one form the mouthpiece of the device of the present invention is an integral subassembly. In another form the mouthpiece is made of two or more components which can be assembled together. In one form the mouthpiece is demountable from the canister. In another form the mouthpiece is integral with the canister.

It is to be noted that the mouth piece can be used in any application requiring a sealing mouthpiece where there is a flow of air, gas, fluid or the like using the mouth and where it is desirable that little or no leakage of the gas, fluid or the like take place during use, such as for example, in inflating pneumatic devices, including balloons, air or inflatable toys, furniture, airbeds, respiratory devices or the like.

Typically, the mouthpiece includes a bite element. One example of the bite element is a bite block, bite stick or the like. Typically the bite block is adapted for engagement with the teeth of a person, preferably being able to be gripped by the front teeth or front incisors, to securely hold the mouthpiece between the teeth to secure the mouthpiece within the mouth during use, and to assist in sealing the mouthpiece to the mouth. Typically, the bite block has a rectangular shape and/or configuration.

Typically the mouthpiece includes a lip seal element or profile. In one form the lip seal element is a generally arcuate rib or other projection or enlargement. In one form the rib or projection curves from a trough at the front and rear of the mouthpiece to a crest located at or towards either side of the mouthpiece. More typically, the rib is a continuous loop, ring, projection or the like. More typically, the position or location of the trough of the continuous lip sealing rib corresponds to the position of engagement of the front teeth with the mouthpiece and/or sealing element and the location or position of the crests at the sides of the lip seal rib correspond to the position of the corners of the mouth of the person using the mouth piece, particularly to administer the medicament. More typically the side edges, or shorter lateral sides of the rectangular bite block correspond to the position of wings provided on the lip seal ring for being received within or engaging with the corners of the mouth. Typically, the cross-section, profile or section of the lip sealing ring is curved, radiused, semi-circular, part circular, smooth sided or the like.

Typically, all or part of the bite block extends outwardly from part of the lip seal ring. More typically, the lip seal rib surrounds the bite block and the bite block is located internally within the lip seal ring. Additionally, the bite block is located distally of the position of the lip seal ring.

In one form the bite block defines one end of an outwardly extending, or outwardly directed delivery tube or passage through which air flows in use of the mouthpiece when exhaling and/or inhaling. More typically, the interior passage or surface of the delivery tube is provided with a surface treatment, including a chemical or physical surface treatment, such as for example, dimples or other surface treatment or irregularities as is the interior of the mouthpiece and/or canister or other internal walls of the device provided with a similar surface treatment to reduce the friction and/or to reduce static build up on the interior surface so that a full dosage of medicament, preferably in the form of a powder or similar is delivered through the mouthpiece without a significant amount of the medicament remaining as a residue or similar within the device, such as for example, remaining in contact with the interior wall surface of the delivery tube, mouthpiece, canister and/or other interior walls of the various sub-assemblies of the dosage device.

Typically, the mouthpiece including the delivery tube is rigid or semi-rigid. Furthermore, it is preferable that the mouthpiece, and/or bite block not be made of rubber or other resilient or flexible or readily deformable materials but be made of a material which substantially retains its shape, structure, integrity or the like, so as to provide resistance to movement, deflection, deformation or the like to enable the mouthpiece and/or bite block to be gripped between the teeth.

Typically, the tube extends more or less perpendicularly and/or obliquely or outwardly from the body of the mouthpiece.

More typically the end of the mouthpiece is angularly inclined outward or bevelled, tapered, angled, mitred or the like so as to form or be combined with a tongue depressor to keep the tongue away from the open airway thereby allowing the full dosage of medicament to be delivered through the mouth to the airways or air passage including the lungs without the flow being hindered by impinging on the teeth, tongue, back of the throat, or other parts of the oral cavity or the like.

Typically, the mouthpiece is provided with a cap or cover portion. More typically, the delivery tube is located centrally on the cap or is located offset on the cap.

Typically, the profile of the lip seal ring or rib is of variable thickness so that it is thicker towards the upturned edges located at or towards the side of the tube in the form of wings for engaging with the corner of the mouth and is of less thickness in the trough engaging with the middle portion of the lips in the region where the front teeth engage the bite block. However, other variations of thickness are possible.

Typically, the mouthpiece of the present invention is provided with an internal collar arrangement. More typically, the internal collar is a sealing collar for contacting the flap of the valve to assist in directing flow of air during use of the device.

In one form the collar seals the vents during inhaling so as to direct flow internally within the collar and into the mouthpiece during inhaling or inspiration. Typically, the outer edge of the collar or annular rib extends axially from the mouthpiece to a greater amount than the side flange of the mouthpiece so as to penetrate further into the canister to contact the valve seated in the end of the canister to assist in directing exhaled air away from the canister to be vented to atmosphere.

Typically, the mouthpiece is provided with a mask for fitting over the end of the mouthpiece and/or over the mouth and nose of the person using the device. More typically, the mask fits over the bite block and/or around the lip sealing rib or ring to contact an intermediate part of the tube thereby allowing the mouthpiece to be used by children, the elderly, the infirm, the disabled, the mobility impaired, or persons having reduced function, particularly of the hands, mouth or the like.

Typically, the mouthpiece of the present invention can be used with the storage space of the present invention in the form of the canister or chamber of the dosage device of the present invention or can be fitted to filter devices or spirometry devices or other medical devices used in respiratory investigations, monitoring and/or treatments or the like. The mouthpiece of the present invention can be used in any circumstance where a mouthpiece is required, such as for example, when inflating an air bed, inflatable toy, or other device or product requiring the inflow or outflow of air, gases, fluids or the like, such as in lung function testing or treatment, including inspiration and respiratory devices.

The present invention relates generally to inhalation devices for use in delivering a measured dose of medicament to the airways of a person suffering from a bronchial condition and to methods of using such devices to administer the medicament.

In one aspect the present invention relates to a dosage device for delivering medicament to, and/or through the mouth of a person in which the dosage device is provided with a subassembly for preventing cross-contamination of the device by different people using the device or by the same person reusing the device.

In one aspect the present invention relates to a delivery system for delivering a predetermined amount or dosage of a medicament or medication to a person in which the delivery system includes two at least partially separate air pathways that prevent air being introduced into the device and air being expelled from the device from mixing with each other in the dosage containing part of the device so that the expelled air cannot re-enter the device to contaminate the device, particularly the part of the device temporarily containing the medicament immediately prior to delivery to the person.

In one aspect the present invention relates to a mouthpiece adapted for sealing to the mouth and/or lips of a person using the mouthpiece, particularly for connection to or for use with a testing device, treatment device, monitoring device or the like including a delivery or dosage device to assist in delivery of a full dose of medicament to the airways of a person using the mouthpiece.

The present invention finds particular application as a dosage device or delivery system for delivering a measured amount of medicament to a person by inhaling through the device wherein the device is provided with a sealing mouthpiece that minimises the amount of air and/or medicament escaping from the mouth of a person using the device and wherein the device is provided with a chamber having vents or ducts for directing expelled air or breath exhaled from the person away from the device so as to be vented or exhausted to atmosphere thereby reducing the chances of the interior of the dosage device being contaminated by the expelled breath.

The present invention also relates to a method of using the dosage device to deliver a substantially full or complete amount of a measured amount or dose of therapeutic agent or medicament to a person by the person inhaling the medicament through the device and mouthpiece wherein the mouthpiece is provided with a sealing arrangement to seal the mouth and the mouthpiece so that there is less loss of medicament from around the edges of the mouth during inhalation, and exhaling wherein the exhaled breath is channelled away from the dosage device through outlets vented to atmosphere thereby preventing contamination of the device by the expelled air.

Although the present invention would be described with particular reference to embodiments of the present invention in which some embodiments are provided with a vented chamber, other embodiments are provided with a modified mouthpiece and still other embodiments are provided with both a modified mouthpiece and vented chamber, it is to be noted that the scope of the invention is not limited to the described embodiments but rather the scope of the present invention is more extensive so as to include other forms and variations of the dosage device, of the delivery system, of the components forming the dosage device, including the vented chamber and modified mouthpiece and of the methods of using the various forms and variations of the dosage device or delivery system in a variety of different applications not restricted to delivering a substantially full amount of the measured dosage of medicament and/or to avoiding cross contamination of the dosage or delivery device.

One form of the present invention, as illustrated in the drawings, will now be described in detail.

The dosage device of the present invention, generally denoted as 2, comprises three main parts or subassemblies which are a canister or hollow chamber, generally denoted as 10, a one way valve arrangement, generally denoted as 40, locatable at or about the open end of canister 10 and a mouth piece assembly, generally denoted as 60, connected to the open top end of canister 10. In use, the dosage device 2 is fitted to a conventional inhaler, such as a metered dose inhaler (MDI) (not shown) of the type having a pressurised inactive gas that propels a measured dose of medicament or therapeutic agent, such as for example, a bronchodilator, in each “blast” or “puff” produced by activation of the MDI by pressing the top of the MDI to release the pressure within the MDI to dispense the contents, such as for example, a suitable medicament for treating a person suffering from asthma. Once the MDI is pressed or activated, a measured dose of medicament contained under pressure within the MDI is released from the MDI and is admitted to the canister 10 from which the dose can be delivered through the one-way valve 40 located within the dosage device 10 by a person inhaling through the mouth piece 60 as will be described in more detail later in tis specification.

One form of the canister subassembly 10 will now be described in detail. Other forms including other shapes, arrangements, configurations and types are possible. It is to be noted that the primary function of canister 10 is to act as a reservoir or temporary container for the medicament or therapeutic agent immediately prior to administering the dosage of medicament to the person using the dosage device.

It is to be noted that the canister 10 can take many different forms with different combinations of features. One form of the canister will now be described.

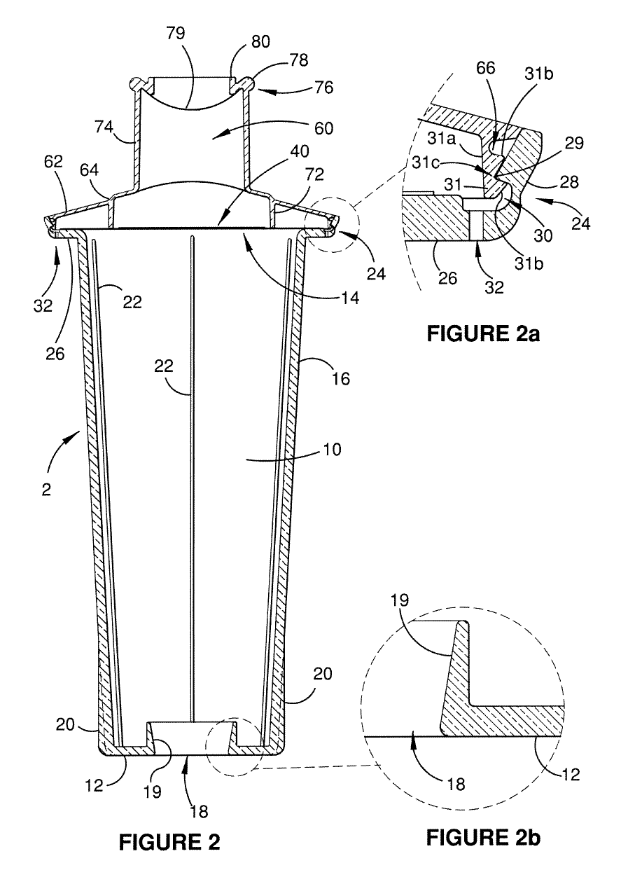

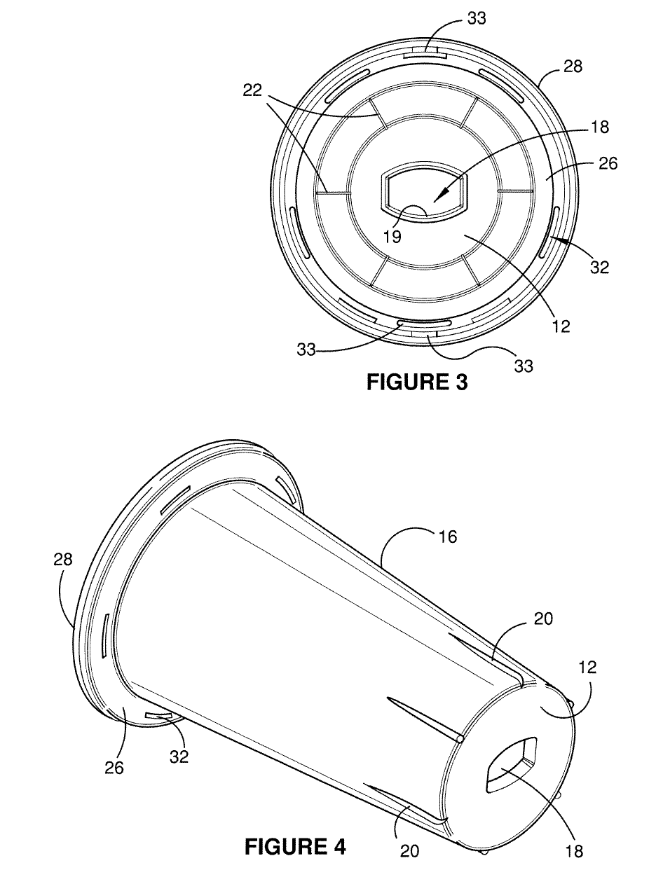

In one form canister 10 is a substantially hollow, cylindrical body or chamber having a tapering side and is provided with a relatively smaller diameter open circular base 12 located at one end which is the inlet end for admitting the medicament internally into canister 10 and an open circular top 14, of relatively larger diameter located at the other end which is the discharge and/or delivery end of the canister connected by a tapering wall 16 extending from smaller diameter base 12 to larger diameter top. Base 12 is provided with a shaped aperture 18 for sealingly receiving therein the discharge chute or dispensing end of an MDI such as the standard MDI inhaler. The shape of the aperture 18 corresponds to the shape of the discharge chute of the MDI which is generally a quadrilateral having curved or arcuate sides or similar. Aperture 18 is provided with an internal wall profile 19 extending generally axially into the interior of canister 10, typically a sloping or tapering wall profile 19, particularly a backwards or reverse tapering sloping wall profile, as shown in FIG. 2 to sealingly receive therein the delivery chute of the MDI. The length of wall 19 is of sufficient height to prevent excessive radial movement of the MDI inhaler when inserted into aperture 18 and is of a length and shape to form a seal between device 2 and the MDI inhaler.

It is to be noted that the dosage device 2 can be provided with an optional adaptor (not shown) for coupling or connecting the dosage device to the MDI rather than using aperture 18 to receive the inhaler directly within. The adaptor can take any suitable or convenient form and can be of a one piece construction or of a two piece construction. The purpose of the adaptor is to couple or connect the MDI inhaler to dosage device 2, more particularly, to the interior of canister 10.

One or more linear projections 20 extend lengthwise along part of tapering side wall 16 of canister 10 from base 12 to part wall along side wall 16 to prevent canister 10 or dosage device 2 from rolling about the central lengthwise extending axis when placed on its side. Alternatively, canister 10 can be provided with one or more flat segments (not shown) located at spaced apart locations around the perimeter of canister 10 to stop the canister from rolling on its side 16. The canister can have any number of linear projections 20 as required or thought appropriate. Projections 20 can be used to help a multitude of canisters to be stacked one within the other, such as, to nest one within the other and to allow easy separation one from the other.

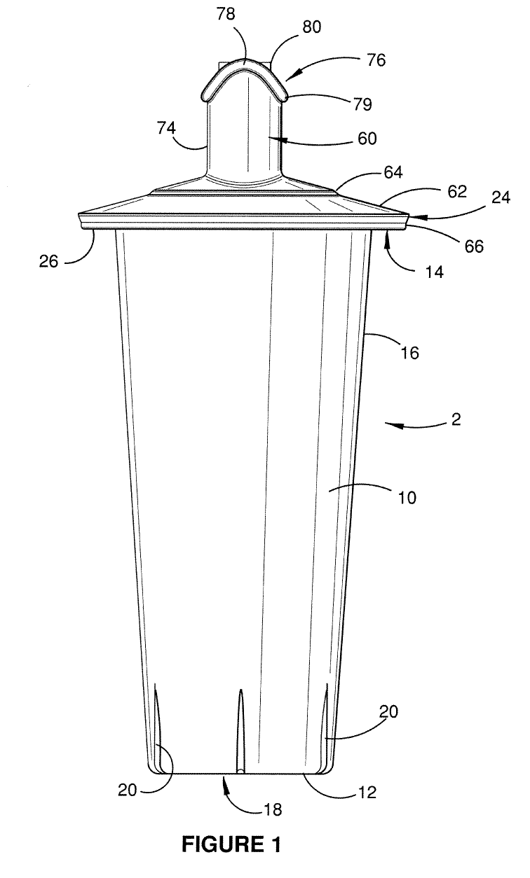

Additionally, the external longitudinal projections 20 extending lengthwise along the lengthwise extending axis of canister 10 are provided also to prevent or reduce the incidence of the tapering wall 16 being scratched or scratch marks appearing on the wall of the canister during stacking of adjacent canisters, such as for example, nesting of the canisters, during the manufacturing and/or assembly processes of making/packaging of the dosage devices. Longitudinal ribs 20 may extend along the entire length of wall 16 or partially along wall 16, particularly near to base 12 as shown in the drawings, particularly FIGS. 1, 4 and 5. Each rib 20 can be continuous or may be formed into separate spaced apart sections or segments. Preferably, there are six spaced apart ribs 20 located at evenly spaced apart locations around the external side wall circumference of canister 10. However, any number of ribs 20 can be present.

Additionally, lengthwise extending internal ribs 22 are provided to extend substantially axially along the inner surface of wall 16 of canister 10 from the top of canister 10 to the base 12 of canister 10. Internal ribs 22 are provided to absorb or smooth any surface imperfections or irregularities that may appear during manufacture of canister 10, such as for example, in internal side wall 16 when moulding canister 10 so as to provide a smooth internal surface free from distortion to assist in delivering a full dose of medicament and/or to assist stacking and/or nesting of a multitude of similar canisters, one within the other. Additionally, internal ribs 22 assist in maintaining the inner surface of canister 10 free from scratches, abrasions or the like during stacking of two or more canisters, such as for example, during or after the manufacturing process by preventing contact of the inner surface of wall 16 of one canister with the outer wall of another canister when one canister is received inside the other.

Open circular end 14 of canister 10 is provided with a sealing arrangement, generally denoted as 24 formed from the interconnection of the external surface of the side wall of canister 10 with the internal surface of the side wall of mouthpiece 60. Sealing arrangement 24 can take one of many different forms. One such form will now be described. Further it is to be noted that one end of mouthpiece 60 is provided with a complementary sealing arrangement to the upper open end of canister 10 so that canister 10 and mouthpiece 60 can be fastened together with the respective sealing arrangements cooperatively engaging with one another to seal the mouthpiece to the canister to prevent air flow past the seal, preferably in both directions, i.e. radially inwards and radially outwards.

Sealing arrangement 24 comprises canister 10 having a radially outwardly extending circumferential rim or flange 26 extending substantially perpendicularly radially outwardly from the top of tapering wall 16 around open top 14 of canister 10 and an upstanding flange 28 surrounding rim 26 arranged to extend in a substantially upwards and outwards direction to rim 26. An undercut circular groove 30 having a generally curved profile is located circumferentially around sealing arrangement 24 at the junction of rim 26 and flange 28 for receiving therein a downwardly depending flange 31 of mouthpiece assembly 60. Flange 28 is provided with projection 29 arranged to extend radially inboard. Undercut groove 30 forms a channel or conduit for receiving and directing exhaled air from mouthpiece 60 for discharge from dosage device 2 by being in fluid communication with mouthpiece 60 when the valve is in the closed position. Flange 31 in one embodiment, is provided with a smooth or straight inner side 31a and a complex profile on the outer side including two protruding or projecting sections 31b having a trough 31c located therebetween for receiving a protruding part 29 of flange 28 therein to form the seal between flange 28 of canister 10 and flange 31 of mouthpiece 60.

Optionally, two lugs 33 (not shown), project upwardly and outwardly from rim 26 to extend into groove 30 to join flange 28 to form two locating lugs for correctly aligning mouthpiece 60 on to the open end of canister 10. In one form, mouthpiece 60 is provided with complementary cut-outs 70 (to be described later), for receiving the locating lugs to correctly orientate the mouthpiece on the canister. The lugs 33, if present, are located diametrically opposite one another. It is to be noted that any suitable number and orientation of lugs 33 can be used.

A multitude of vents in the form of elongated apertures, openings, slits, slots 32 or the like are provided at regularly spaced apart locations in rim 26 to form air vents for allowing air or exhaled breath or gases to be expelled from canister 10 during use, such as when a person using device 2 exhales, so as not to contaminate the interior of canister 10 as will be described in more detail later. Any number of slots 32 located at regularly spaced apart locations around the circumference of device 2 can be used. Further, it is to be noted that slots 32 can take any suitable shape, orientation or style. In use, slots 32 are in fluid communication with the channel formed by circumferential groove 30 so that exhaled air directed into groove 30 flows through groove 30 to exit through vents 32.

One or more ridges such as projection 29 can also be located in flange 28 provided on rim 26 to extend upwardly from the surface of rim 26 to assist in locating mouthpiece 60 on the open end 14 of canister 10 such as by the mouthpiece 60 snap fittingly engaging with one or more of the ridges to releasably secure mouth piece 60 to canister 10. Optionally, one or more of the ridges are located at circumferentially spaced apart locations around the perimeter of rim 26. The ridges can be continuous or can be arranged in segments or sections. In one form the ridges are generally arcuate in shape and are located intermediate two adjacent vents 32. The number of ridges can be the same as or different to the number of vents 32.

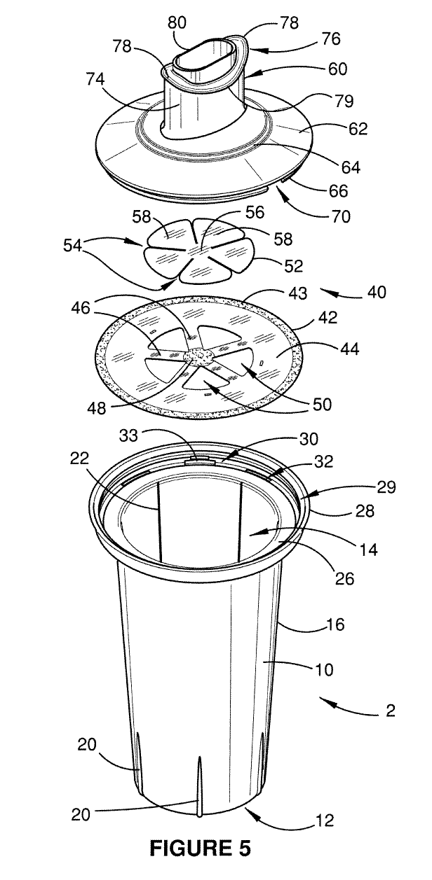

With particular reference to FIG. 5, one form of one way valve arrangement 40 will now be described. This form of the valve is a diaphragm valve which is sealingly located on the open end 14 of canister 10 to selectively close the open end 14 of canister 10 in accordance with the direction of airflow through dosage device 2. One form of the diaphragm valve will now be described. The valve 40 comprises a generally perforated circular disc in the form of a diaphragm skeleton or web 42. Web 42 comprises a solid generally annular outer section 44 and a multitude of radially extending spokes 46 extending radially from the inboard edge of outer annular section 44 to a solid central hub 48 of the perforated disc web 42. Generally sector shaped apertures 50, preferably generally triangularly shaped apertures, are formed in the intermediate section of web 42 between consecutive spokes 46 so that the apertures and spokes are alternately arranged with respect to each other. In one form, web 42 is in the form of a 5-spoke wheel-like structure. However, the shape of the web, the shape of the spokes and the shape of the apertures may be varied to suit requirements including having different numbers of spokes, apertures or the like. The diameter of web 42 is such so that the web 42 can be received within the open end 14 of canister 10 to rest upon rim 26 to seal the open end 14, but allow air to pass through to vents 32.

The apron or outer edge 43 of web 42 is sealingly connected to the inner periphery of rim 26, such as by thermal bonding, sonic welding, or other suitable adhesive or bonding agent to locate valve 40 and to assist in operation of device 2. In one form, outer edge 43 is releasably bonded to rim 26. A generally circular sector shaped diaphragm flap 52, such as in the form of a five leaf clover or similar, forming a flexible substantially solid disc is located over the intermediate part of web 42 containing apertures 50 and central hub 48. Flaps 52 are each of a shape and size sufficiently large enough so as to cover all of one of the apertures 50 in web 42 so as to be able to seal collectively the apertures in use when pressed against web 42 by a person using the device exhaling. However, flap 52 is sufficiently flexible so as to deform or deflect slightly in use of device 2 when a person is inhaling to allow air and entrained medicament to pass from canister 10 through apertures 50 of valve 40 into mouthpiece 60 for delivery to the oral cavity of the person using the dosage device. Typically, web 42 and flap 50 are made from different thicknesses of polypropylene or other suitable material. A preferred material is Tyvek™ made by Du Pont.

Flap 52 is provided with a multitude of slits 54 extending radially inwards from the outer edge of flap 52 towards central hub 56 which is solid to define five movable sectors 58 or panels so that the slits 54 and sectors or panels 58 are alternately arranged. Slits 54 allow each of the sectors 58 to deflect so as to move between an open position and a closed position independently of one another. It is to be noted that each slit 54 is aligned to extend along each of the spokes 46 of web 42 so that the slits 54 are sealed against spokes 46 when the valve is in the closed position during exhaling or expelling air.

In one form, valve 40 and canister 10 may be a single piece with web 42 formed integrally with canister 10 or maybe of a two piece construction with valve 40 removable from canister 10 so as to replace valve 40 as required or to clean canister 10 periodically. Flap 52 is secured to web 42 by any suitable adhesive or bonding means such as for example, by a suitable adhesive or bonding agent located between centre 56 of flap 52 and hub 48 of web 42.

In one form flap 52 is fixedly connected to hub 48 of web 42 whereas in other forms flap 52 is connected to spokes 46 of web 42 whereas in still other forms flap 52 is connected to both hub 48 and spokes 46 of web 42. Further it is to be noted that the radially outboard edges of each of segments or sectors 58 of flap 52 are free to separate from web 42 to allow passage of air through valve 40 by deflecting or deforming away from the surface of web 42 such as when air is inhaled through mouthpiece 60. In use, when a person exhales sectors or segments 58 of flap 52 are forced into contact with web 42 so as to seal apertures 48 to direct the exhaled breath to the perimeter channel formed by groove 30 around the periphery of canister 10 for discharge through vents 32 to expel air from device 2. When a person inhales, sectors or segments 58 of flap 52 are drawn away from web 42 or spokes 46, thus opening apertures 50 allowing air to pass therethrough for movement into and through mouthpiece 60 so as to administer the medicament to the person using the dosage device.

One form of mouthpiece 60 will now be described in detail. However, it is to be noted that other forms of the mouthpiece are possible. Further, although the mouthpiece is primarily designed for use with dosage device 2, the mouthpiece, particularly that part of the mouthpiece received in the mouth, can be used in a variety of different applications for a variety of purposes.

In one form, mouthpiece 60 comprises a generally circular cap 62 having a side wall sloping radially from being raised at or towards the centre of the mouthpiece being relatively lower around the circular perimeter of the edge of the mouthpiece. One or more circular ridges 64 are formed in the upper surface of the sloping wall of cap 62. A downwardly depending flange 66 is provided circumferentially around the edge of cap 62 extending axially from circular edge. A circumferential aligning nib or finger (not shown), is located at the outboard edge of flange 66 for aligning with arcuate ridges if present on canister 10 to assist in accurately aligning mouthpiece 60 and canister 10 in register with each other and snap-fittingly attaching mouthpiece 60 to canister 10 in use. If present, the aligning nib extends outwardly from flange 66 and is provided with a bevelled edge for being received in groove 30. Alternately, cap 62 is provided with a cut-out 70, for receiving a complementary lug 33 therein to align mouthpiece 60 in register with canister 10 when being fitted to the top 14 of canister 10. Other forms or arrangements for aligning mouthpiece 60 with canister 10 prior to connecting the two subassemblies together are possible.

In one form mouthpiece 60 is provided with a pair of cut-outs 70 which are located diametrically opposite one another in downwardly depending flange 66. The spacing of cut-outs 70 correspond to the spacing of lugs 33 of canister 10, if present, to assist in accurately locating mouthpiece 60 on canister 10 in the correct orientation. A downwardly depending internal collar 72 is located on the inner surface of sloping wall of cap 62 inboard of flange 66 for sealingly engaging with web 42 of valve 40 to assist in sealing valve 40 when a person exhales. The diameter of circular collar 72 is slightly larger than the diameter of flap 52 so that the edge of collar 72 contacts web 42 but not flap 52 so that flap 52 is located within circular collar 72 and thus the segments 58 are free to move between the closed position and open position to close and open valve 40. Thus, collar 72 or annular rib seals off the outlet vents 32 of canister 10 during inhaling to assist in delivering a full dose of medicament from canister 10 without loss of air and entrained medicament from the canister prior to delivery into mouthpiece 60.

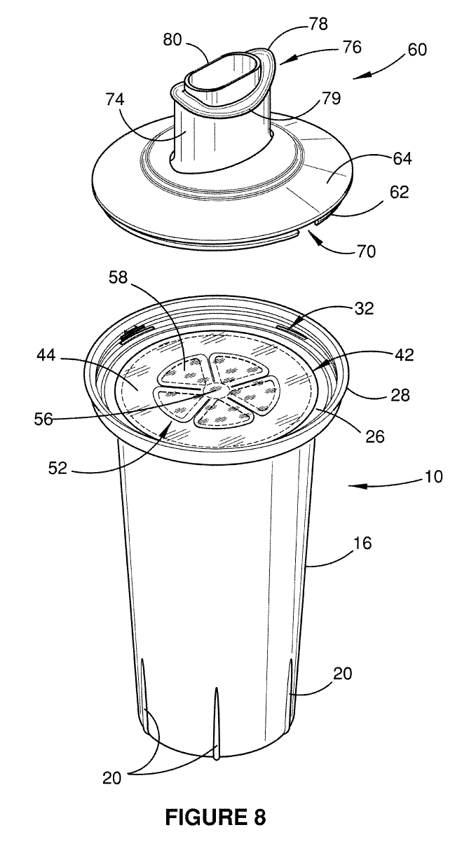

A generally curved shaped open ended tubular stem 74 having an open end at both ends is provided to extend outwardly from the centre of the outer surface of cap 62 to form a delivery tube for delivering medicaments to a person using the device 2 when admitted to mouthpiece 60. The inboard end of delivery tube 74 is located wholly within collar 72 as shown more particularly in FIG. 7. In one form, tube 74 is elliptical in profile or cross-section having a major axis and a minor axis substantially perpendicular to each other. Other suitable arcuate shapes or curved shapes of delivery tube 74 are possible to fit comfortably within the mouth, such as circular, oval, round, or the like. The external surface of the distal end of tube 74 is provided with a lip sealing ridge 76 in the form of an undulating continuous loop or ring around stem 74 for engaging with the lips of a person using the device to seal the lips to the device to ensure that no air or very little air escapes between the lips when inhaling so that a full dose of medicament is delivered from canister 10 into the mouth when a person inhales. Lip sealing ridge 76 is curved in cross-section in both the horizontal and vertical directions by being undulating having relatively high points or crests 78 at either side edge almost in the form of wings, and relatively low points or troughs 79 located between the high points forming the side edges so as to be closely shaped to the shape and profile of the mouth and/or lips so as to effect a seal in use by the mouth being received over the lip sealing ring so that the ring is in contact with the inside of the mouth.

Stem 74 terminates at or towards its distal end in a bite element in the form of a bite block 80 of a generally square or rectangular shape having curved or rounded or radiused corners located wholly within the profile of the lip sealing ridge 76 for contact with the teeth of a person using device 2. Bite block 80 is of a smaller width and of a smaller length in the horizontal or transverse plane than the corresponding width and transverse length of the lip sealing ridge 78 and extends distally outwardly therefrom as illustrated in the drawings so as to be contactable by the teeth and/or tongue when located in the mouth. Further, it is to be noted that the longer transverse axis of the bite block 80 extending from near to the crests 78 on either side of lip sealing ridge 76 is aligned along the major axis of the elliptical tube 74 and of the lip seal ring 76 and the shorter axis of the bite block 80 is aligned along the minor axis of the tube 74 and of lip seal ring 76 extending from trough to trough 79. In use, bite block 80 can be clamped between the teeth, particularly the front incisors, to securely hold the mouthpiece within the mouth during use and the central part of the lips can be located over the central part of the lip sealing ring 76 and the outer parts of the mouth can be located over the ends or wings 78 of the lip sealing ring 76 to completely seal the end of the mouthpiece within the mouth. The lips of the person also engage the troughs 79 of ring 76 to completely seal the mouthpiece in the mouth.

In one form of device 2 of the present invention, a MDI inhaler (not shown) is sealingly fitted into aperture 18 at the open base end 12 of canister 10 so that the walls of the delivery chute of the MDI fits snugly against wall 19 of aperture 18 to securely retain the MDI to device 2 to enable a full dose of medicament to be delivered to canister 10 when the top of the MDI is pressed. The full dose of medicament is held temporarily in the canister 10 until a person inhales through mouthpiece 60.

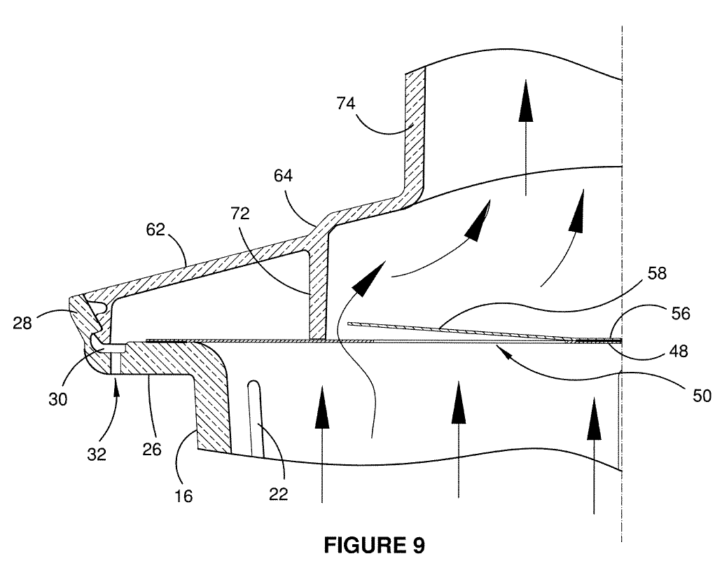

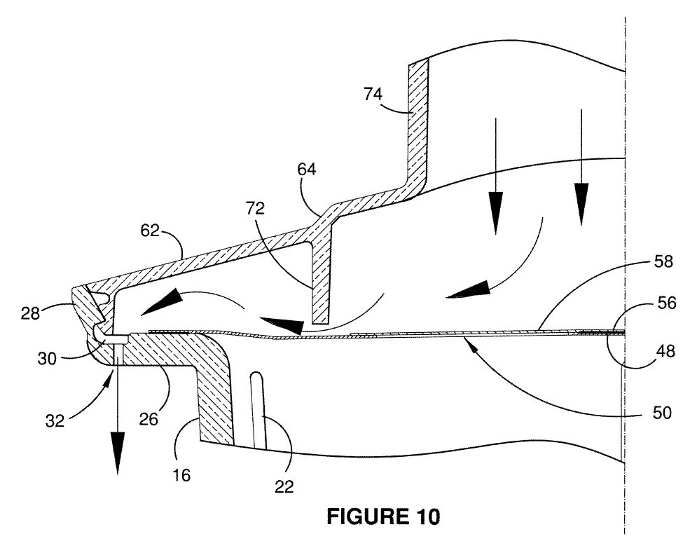

A person locates mouthpiece 60 in the mouth with bite block 80 securely clamped between the front teeth and the lips sealingly engaged around and over the lip seal ring 76 to seal the end of mouthpiece 60 to the mouth. On inhalation, the segments 58 of the flap 52 are withdrawn away from web 42 by the partial vacuum formed in mouthpiece 60 to allow air containing the medicaments to be withdrawn from canister 10 through valve 40 into mouthpiece 60 and into the mouth of the person for administration directly to the lungs along the flow paths illustrated by the arrows as shown in FIG. 9. As the mouth and lips are sealed to the mouthpiece to prevent escape of air, a full dose of medicament is drawn into the mouth for administration to the lungs of the person on the person inhaling. The person holds their breath for a short period of time to allow the full dose of medicament to be deposited and/or consumed, where required, and to fully enter the airways and air passage to relieve the adverse effects of asthma or the like.

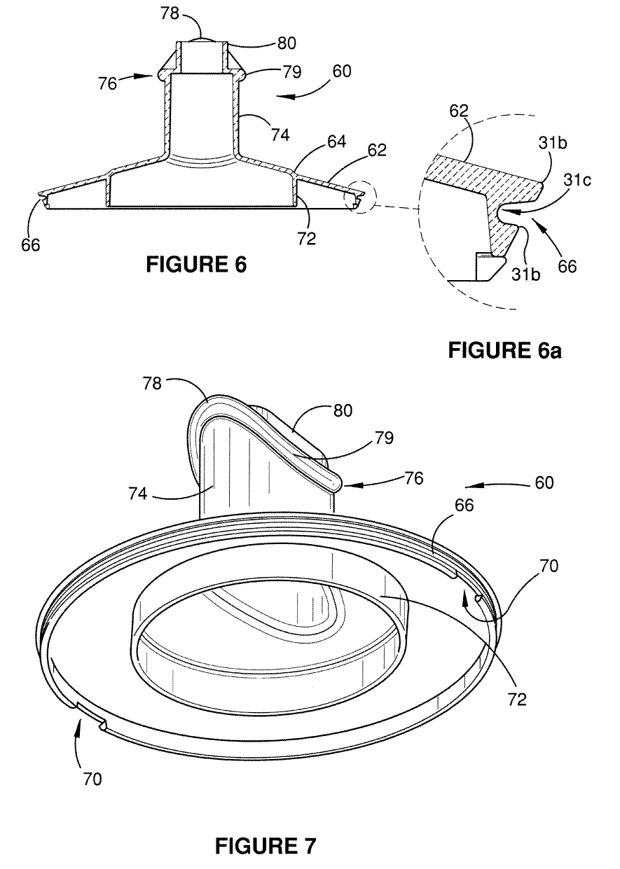

After the full dose has been administered, whilst the mouthpiece is sealingly located in the mouth of the person between the lips, the person exhales through the mouthpiece whereupon the mouthpiece is pressurised to an extent that the segments 58 of flap 52 are forced against web 42 to close valve 40 and to deflect the top of segments 58 away from the lower edge of internal collar 72 to allow air flow past internal collar 72, whereupon the flow of air is directed over flap 52, under collar 72, to and through the channel formed by groove 30 along the flow paths indicated by the arrows of FIG. 6. The exhaled air is forced through vents 32 in fluid communication with groove 30 for discharge to the outside of device 2 by being vented to atmosphere. As valve 40 is in the closed position no exhaled breath can enter canister 10 to contaminate canister 10 or its contents thus allowing canister 10, and device 2, to be reused without having to clean canister 10 and thus no contamination or cross-contamination can occur by exhaled air mixing with medicament or air in canister 10.

### ADVANTAGES OF THE PRESENT INVENTION

Advantages of the present invention include the following:

As the one way valve prevents exhaled breath or gases from passing through the valve into the canister, the canister of the present invention can not be contaminated nor can its contents be contaminated with the result that it is not necessary to clean the canister after every use even if different people are using the one canister since there is no risk of cross infection between the subsequent users as the exhaled breath or gases are discharged to the outside of the device and to not contact the inside of the device.

The inclusion of antistatic devices in the form of dimples or other surface irregularities on the internal walls of the subassemblies or anti static additives in the material from which the mouthpiece and/or canister are manufactured prevents particles of the medicament from adhering to the internal walls of the canister or mouthpiece thereby ensuring that a full dosage of medicament is delivered to the person.

The presence of the bite block at the end of delivery tube of the mouthpiece keeps teeth out of the way so that the medicament particles do not impinge upon the teeth where they could remain thereby delivering a less than full dose of the medicament to the lungs. By removing the teeth from the direct flow path of the medicament the full dosage of medicament is delivered to the lungs.

The presence of the bite block not only allows the mouthpiece and device to be securely connected to the mouth but that the one size of bite block can be accommodated within a wide variety of different sized mouths so that the smaller mouths of children can be sealingly connected to the end of the delivery tube to deliver a full dose.

The use of the bite block and lip seal ring in combination provides a better seal between the mouth and the mouthpiece and additionally keeps the teeth away so that the airflow from the end of the mouthpiece to the oral cavity is not impeded by particles striking the throat or teeth before being administered to the lungs of the person.

The presence of the bite block and lip sealing ring also results in the nose of the person using the dosage device to be located slightly away from the end of the mouthpiece or the delivery tube so that the dosage device is more comfortable to use and can be positioned optimally to deliver the full dose.

The dosage device of the present invention allows the one canister to be shared between a number of different users, each with their own mouthpiece, because there is no danger of the canister being contaminated or cross-contaminated.

The presence of flat segments or longitudinal ribs on the external surface of the canister may prevent the canister from rolling, such as for example, from rolling off the bench or table and falling to the floor where it could be contaminated.

The described arrangement has been advanced by explanation and many modifications may be made without departing from the spirit and scope of the invention which includes every novel feature and novel combination of features herein disclosed.

Those skilled in the art will appreciate that the invention described herein is susceptible to variations and modifications other than those specifically described. It is understood that the invention includes all such variations and modifications which fall within the spirit and scope. Accordingly, it is to be understood that the above description is intended to be illustrative and not restrictive. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

All terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

We claim:

1. A mouthpiece capable of being used to direct a flow of air comprising a first end having a bite element for engaging with the teeth of a person using the mouthpiece to assist in securely maintaining one end of the mouthpiece within the mouth during use of the mouthpiece, and a lip sealing element located at or towards the first end of the mouthpiece for cooperatively engaging with the lips of a person using the mouthpiece to seal the mouthpiece to the mouth so that during use of the mouthpiece, there is reduced amount of leakage of air flow from the lips and/or mouth of a person using the mouthpiece to inhale and/or exhale.

2. A mouthpiece according to claim 1, in which the mouthpiece includes a bite element in the form of a bite block, bite stick or similar in which the bite block is adapted for engagement with the teeth of a person.

3. A mouthpiece according to claim 1, in which the mouthpiece is provided with a lip seal element or profile.

4. A mouthpiece according to claim 3, in which the lip seal element or profile is substantially curved being in the form of a rib or projection curved around the bite block.

5. A mouthpiece according to claim 1, in which one end of the mouthpiece is angularly inclined outward or bevelled, tapered, mitred or the like so as to form or be combined with a tongue depressor to keep the tongue away from the open airway thereby allowing the full passage of medicament to be delivered through the mouth of the airways or air passages.

6. A mouthpiece according to claim 1, in which the mouthpiece is provided with an internal sealing collar for contacting the flap of the valve for assistance in directing flow therethrough during use of the device.

7. A mouthpiece capable of being used with an inhalation device to introduce a medicament to the oral cavity of a person using the inhalation device through the mouthpiece, the mouthpiece having a first end provided with a bite element for engaging with the teeth of a person using the mouthpiece to assist in securely maintaining the first end of the mouthpiece within the mouth during use and a lip sealing element for cooperatively engaging with the lips of a person to substantially seal the first end of the mouthpiece to the mouth and a second end connected to or connectable to the inhalation device wherein the first end is in fluid communication with a second end so that when the teeth are used to hold the bite element and lips engage with the lip sealing element to seal the first end of the mouthpiece to the mouth, medicament within the inhalation device can be withdrawn through the mouthpiece into the mouth of the person into the mouthpiece so that substantially all of the dosage of medicament is delivered to the person.

8. A dosage device capable of assisting in delivering a measured dose of a medicament to a person using the dosage device, said dosage device comprising a storage chamber for at least temporarily storing a measured dose of a medicament after introduction of a medicament into the chamber, the storage chamber provided with an inlet for admitting the measured dose of a medicament to the storage chamber and a mouthpiece portion adapted for cooperation with the mouth of a person to withdraw the measured dosage of medicament into the mouth of a person upon inhalation by the person wherein the mouthpiece is provided with a bite element for engaging with the teeth of the person using the dosage device to assist in delivering substantially all of the dosage to the person.

9. A dosage device according to claim 8, wherein the dosage device is a one-piece unit or can be made of two or more individual pieces or sub-assemblies which can be connected together or assembled to form the dosage device.

10. A dosage device according to claim 8, in which the dosage device is provided with one or more air vents located at spaced apart relationship for directing air away from the interior of the storage space by exhausting air to atmosphere.

11. A mouth piece for use in connection with an inhalation device comprising at least a delivery tube capable of connection to a source of medicament forming the main body of the mouthpiece, a bite element located at or towards one end of the delivery tube adapted for engagement with the teeth of a person using the mouthpiece and a lip sealing element or profile located around the tube for sealingly engaging with the lips and/or mouth of a person to substantially seal the mouthpiece to the lips and/or mouth wherein the bite element assists in locating the end of the mouthpiece securely within the mouth and the lip sealing element substantially seals the mouth to assist in allowing substantially all of the measured dose of the medicament to be delivered to the airways of a person through the mouth.

12. A delivery system for introducing a medicament into the oral cavity of a person requiring treatment for a bronchial and/or lung condition comprising a storage space for receiving and holding a dose of the medicament prior to introducing the medicament to the airways of a person through the person inhaling, wherein a storage space is provided with at least one vent for directing and/or exhausting air exhaled from the person using the device away from the storage space for discharge from the device away from the storage space so as to vent the exhaled breath to atmosphere thereby reducing or eliminating the chance of contaminating or cross-contaminating the storage space or the contents of the storage space through admitting the exhaled breath to the storage space.

13. A delivery system according to claim 12, further comprising a one way valve arrangement for allowing air to flow in one direction or pathway yet preventing air from flowing in another pathway or direction.

14. A delivery system according to claim 13, in which the valve allows passage of inhaled air from the storage space to the mouthpiece for administration to a person using the mouthpiece and prevents passage of exhaled air through the valve into the storage space after medicament in the storage space has been administered.

15. A delivery system according to claim 14, in which the one way valve includes a web portion for supporting the valve in which the web portion is provided with a plurality of apertures for allowing passage of air or airflow through the valve when the valve is in the open position.

16. A delivery system according to claim 15, in which the one way valve includes a diaphragm having one or more movable flaps such that the flaps are movable between an open position in which the flap is deflected or deformed away from the web portion allowing air to move through the valve and a closed position in which the flap is in sealing contact with the web portion so as to prevent flow of air through the valve or to allow air to be extracted from the device.

17. A delivery system according to claim 16, in which the flap portion of the valve is adhered to the web portion of the valve through respective centre or hub portions such as for example by heat welding, sonic welding, adhesive, bonding agent or similar.

18. A delivery system according to claim 16, in which the diaphragm flap is of a generally circular flat disc having a multitude of segments arranged in side-by-side relationship alternatively with slits allowing the segments to move to open and close the valve.

19. A delivery system according to claim 18, in which the flap diaphragm is provided with slits arranged to extend lengthwise along spokes of the web.

20. A delivery system according to claim 12, in which the storage space is a spacer, reservoir, container, chamber, cavity, canister, vessel, hollow cylinder or like enclosure for more or less temporarily storing a dosage of medicament, particularly a powdered medicament.
